SEQUENCE LISTING (2 sequences)

SEQ ID NO: 1 - DNA, 2820 bp, Propionibacterium granulosum
atgatgaacacctctggccgtatgcgccgggtgctgaccggtatcgccgcggtgacgacctcggccctcgcatgtgccggtctgctcgtgccaagtgccaatgccggcccagctccccacccactcatcaatgaggtctacggtggcggtggcaacagtggagccaccctcacccacgacttcgtcgagctcgtcaatcccacctccgagacgatctcgctggacgggtacgtcgtggagtactacggagccaccggcaatcgcggaaacaggtgcacgttgagcgggtcggtcggtcccggcggtcacttcctcgtccagcaggccaagggagccggtggtacccaggcactgcccaccccggatgccacctgcggtgccaacatgagtggcaccaagggatcggtgcagctggtcggcccggacggcacggtcgtcgacctcgtcggctacggtgccgccgccaaggccgagggaaaaccgaccaaggcactgtcgaacaccacctcggcccagcggggtggcctcgaggacaccgacgacaactccgcggacttcgtcatcggcactcccagcccgatgaactccgccagcacaccggctcccagcacgccaggcactcctgaccagacggccagccccactccgagcggttctgcgacccccactgcctccgagaccccgactggccagacgagcatcgccgacatccagggcaccaccgacaccagcccgatggtcggcaaggtcgtgaccaccacgggcgtcgtcaccgccgtctaccccaagggcgggttcaatggcctctacatccagactcccggctctggtggtgtggccaagaccccgaccgatgcctccgacggcatcttcgtccactccgccaaggccgccagcgcctacaccgcaggtcagtgcgtcgtcgtctccggtgaggtctcggagttcaacggtctcaccgagatcaatgggacgagcgtcaccgagaccaagggctgtgccgacgtcaagccggtgaaactcgcaaccctgccgcgcaccgatgcgcagcgtgaggcgtatgagtccatgctcgtggaaccacagggcacctacaccatctctaacaattaccagctcaaccagttcggcaccgttggcctcgccttcggcaccgagccgctgtaccaaggtaccgaggtggcccgtcccggtgaggatgccaagaaggtcgaggacgagaaccgtgcccgatccatcaatctggacgacggtgcgagctggaactatcagaccaacgacgaggccaagaactccccgctgccgtacctgtcccaggacacaccgatgcgtaccggctcccacgtgggcttcaccagaccggtgatcatggactaccggttcgggtggaacttccagcccactggcatggtctcgggcgcccagtcgccccacagtccgatcaccaccaccaacgatcgtccggccaaggccccgagtttcgactccgatctcaagctcgccagcttcaacgtgctcaactacttcaccgacctgggcaaggacgaggacggctgcaaggcctacacggatcgcaccggcacgccggtcaccgccaatttctgcacggtgcgtggcgcctacacccgagaggcattccacgaccagcaggccaagatcgtcacggccatcaacggtttggacgccgacgtcgtcgcgctcatggaggtggagaactccgcgtccatcacctacctgcccggtcagcctcgtgacaaggccctggcccatctggtggacgaactcaacaaggctgctggttcccaggtctggggatacgtcgcctcgccgaccgtcgtgccgccccatgaggacgtcatccgtaccgcgttcatctaccgactcgactcggtgcgcccggagggaccatcgctcatcctcatggatgatgcctacgccaatgcccgtcagccactggcccagaagttcgtggccaaggacgtcaggggctccttcgtcgccgtcgccaatcacttcaagtcgaagggttccggggaggacgacggcaccggtcagggcaagtcgaacccctcacgtactgtccaggcccaggcactgatcgactggagcgccaaggagttcgccggcgagccggtgttcctgctcggtgacttcaacgcctacggcatggaggatccggtcatgaagatcaaggacgccggctacaccgaggtcgtcgagcagctcgctcctggcgcgtcgaccttccagtacggtggacggctgggttccctggaccacatcttcgccaacgcggcggccatgaggatggtcagtggtgccggggtgtgggacatcaatgccgacgagtccatcgccatgcagtactcccgtcgcaactacaatgtgacggatttccacacgagcggaccatgggctgcctccgaccacgatcccgccctggtggggatcacgttcccgggcgcgtcgaccccgcagcccagtgaaacctcgtccgtcgagccgagcgcgtcggtgtcgacaccgcccagtggtgagccgtctgtttcgcccactgcgtcgcctgccacgccgagtggcgctgtgagtgacgagcccagtgctgctgcgagtgtgacgcctggtaccccggctccgtcgctgactccggttccgtcactgcctggtgggacgcccggcactccgagcgccacaccggcgggggcttcggcatccgcacatggaaccggtactgacgacgtgtcgggggatgccgctcaggctgccgtcgtacctggagggtctgccgatctaccggccctgccggccaccggcatctga

SEQ ID NO: 2 - protein, 939 aa, Propionibacterium granulosum
MMNTSGRMRRVLTGIAAVTTSALACAGLLVPSANAGPAPHPLINEVYGGGGNSGATLTHDFVELVNPTSETISLDGYVVEYYGATGNRGNRCTLSGSVGPGGHFLVQQAKGAGGTQALPTPDATCGANMSGTKGSVQLVGPDGTVVDLVGYGAAAKAEGKPTKALSNTTSAQRGGLEDTDDNSADFVIGTPSPMNSASTPAPSTPGTPDQTASPTPSGSATPTASETPTGQTSIADIQGTTDTSPMVGKVVTTTGVVTAVYPKGGFNGLYIQTPGSGGVAKTPTDASDGIFVHSAKAASAYTAGQCVVVSGEVSEFNGLTEINGTSVTETKGCADVKPVKLATLPRTDAQREAYESMLVEPQGTYTISNNYQLNQFGTVGLAFGTEPLYQGTEVARPGEDAKKVEDENRARSINLDDGASWNYQTNDEAKNSPLPYLSQDTPMRTGSHVGFTRPVIMDYRFGWNFQPTGMVSGAQSPHSPITTTNDRPAKAPSFDSDLKLASFNVLNYFTDLGKDEDGCKAYTDRTGTPVTANFCTVRGAYTREAFHDQQAKIVTAINGLDADVVALMEVENSASITYLPGQPRDKALAHLVDELNKAAGSQVWGYVASPTVVPPHEDVIRTAFIYRLDSVRPEGPSLILMDDAYANARQPLAQKFVAKDVRGSFVAVANHFKSKGSGEDDGTGQGKSNPSRTVQAQALIDWSAKEFAGEPVFLLGDFNAYGMEDPVMKIKDAGYTEVVEQLAPGASTFQYGGRLGSLDHIFANAAAMRMVSGAGVWDINADESIAMQYSRRNYNVTDFHTSGPWAASDHDPALVGITFPGASTPQPSETSSVEPSASVSTPPSGEPSVSPTASPATPSGAVSDEPSAAASVTPGTPAPSLTPVPSLPGGTPGTPSATPAGASASAHGTGTDDVSGDAAQAAVVPGGSADLPALPATGI

(19) United States
(12) Patent Application Publication
(10) Pub. No.: US 2022/0072108 A1
(43) Pub. Date: Mar. 10, 2022

(54) NEW COMPOSITIONS AND METHODS FOR THE TREATMENT OF ACNE VULGARIS

(71) Applicant: VAKONA AB, Umeå (SE)

(72) Inventor: Oleg Alexeyev, Umeå (SE)

(21) Appl. No.: 17/440,371

(22) PCT Filed: Mar. 19, 2020
(86) PCT No.: PCT/SE2020/050290

§ 371 Date: Sep. 17, 2021

(30) Foreign Application Priority Data
Mar. 21, 2019 (SE) 1950356-4

(51) Int. Cl. A61K 38/46; A61P 31/04

(52) U.S. Cl. CPC A61K 38/465; A61P 31/04

(57) ABSTRACT

The present invention relates to the use of a protein, more specifically a P. granulosum DNase, for the treatment and prevention of infectious skin diseases, more specifically to the treatment and prevention of acne vulgaris. The protein is demonstrated to be able to disrupt biofilms formed by pathogenic bacteria, such as biofilm formed by P. acnes.

### FIELD OF THE INVENTION

The present invention relates to the treatment and prevention of infectious skin diseases, more specifically to treatment and prevention of acne vulgaris.

### BACKGROUND TO THE INVENTION

Acne vulgaris is a common inflammatory disorder of the sebaceous follicles, affecting more than 80% of young adolescents, but can also persist into adulthood. Propionibacterium acnes, sometimes also referred to as Cutibacterium acnes, is a Gram-positive pleomorphic rod and is traditionally regarded as part of the normal human skin microbiota and essentially present in the pilosebaceous unit. It plays, together with the sebaceous gland an important role in the development of acne vulgaris.

P. acnes secretes lipases, chemotactic factors, metalloproteases and porphyrins. All interact with molecular oxygen generating toxic, reduced oxygen species and free radicals causing keratinocyte damage and inflammation (Bruggemann. 2005. Insights in the pathogenic potential of Propionibacterium acnes from its complete genome. Semin Cutan Med Surg 24: 67-72).

Biofilm formation is a process during which microorganisms irreversibly attach to and grow on a surface and produce extracellular polymers facilitating adherence and matrix formation. This process results in an alteration of the phenotype of the organisms with respect to their growth rate and gene transcription.

Biofilm formation is considered as a key factor in the pathogenesis of acne (Burkhart & Burkhart. 2007. Expanding the microcomedone theory and acne therapeutics: Propionibacterium acnes biofilm produces biological glue that holds corneocytes together to form plug. J Am Acad Dermatol 57: 722-724.). The biofilm created by P. acnes contributes to the forming of an adhesive glue leading to the binding of corneocytes resulting in micro-comedones. A comedone is a clogged hair follicle or skin pore in the skin. Keratin, or skin debris, combines with oil to block the follicle or pore. A comedone can be open, also referred to as blackhead, or closed by skin, also referred to as whitehead, and occur with or without acne. The chronic inflammatory condition that usually includes both comedones and inflamed papules and pustules, or pimples, is called acne

It has been demonstrated that cells covered with P. acnes biofilm are more resistant to antimicrobial agents compared with planktonic cells, while producing more extracellular lipases. (Coenye et al. 2007. Biofilm formation by Propionibacterium acnes is associated with increased resistance to antimicrobial agents and increased production of putative virulence factors. Res Microbiol 158: 386-392). This finding may explain a certain number of antibiotic therapy failures. Other work showed that biofilm formation by P. acnes was lower when isolated from healthy skin compared with biomaterial-related infections (Holmberg et al. 2009. Biofilm formation by Propionibacterium acnes is a characteristic of invasive isolates. Clin Microbiol Infect 15: 787-795).

A recent case-control study investigated in vivo by biopsies of acne lesions the occurrence and localization of P. acnes on the face and characterized the P. acnes phylotype in 38 acne patients and matching controls: P. acnes within a biofilm was significantly more frequent in acne patients (37% of acne patients compared to 13% of control samples (Jahns et al. 2012. An increased incidence of Propionibacterium acnes biofilms in acne vulgaris: a case-control study. Br J Dermatol 167: 50-58).

Biofilm formation has also been demonstrated in a number of other dermatological disease, such as atopic dermatitis, candidiasis, bullous impetigo and pemphigus foliaceus (Nusbaum et al. 2012. Biofilms in Dermatology. Skin Therapy Letter 17: 7).

As stated in Rumbaugh, et al. (D. Fleming, K. P. Rumbaugh, Approaches to Dispersing Medical Biofilms, Microorganisms 5(2) (2017)) biofilm-associated infections pose a complex problem to the medical community, in that residence within the protection of a biofilm affords pathogens greatly increased tolerances to antibiotics and antimicrobials, as well as protection from the host immune response. Since as much as 80% of human bacterial infections are biofilm-associated, many researchers have begun investigating therapies that specifically target the biofilm architecture, thereby dispersing the microbial cells into their more vulnerable, planktonic mode of life.

Traditionally, infections have been treated by directly targeting the causative pathogens. However, biofilms change the game by providing microbes with greatly increased protection from antimicrobials, causing the effective concentrations to be elevated to dangerous levels. Therefore, some researchers have switched their focus to anti-biofilm agents testing of compounds and strategies that lead to a dispersal event: dispersal agents.

Clinically, dispersal can be accomplished by utilizing enzymes, small molecules, or any other means to trigger a massive dispersal event, either passive or active, that releases the biofilm-associated microbes into their more vulnerable, planktonic state.

As further stated in Rumbaugh, et al. (D. Fleming, K. P. Rumbaugh, Approaches to Dispersing Medical Biofilms, Microorganisms 5(2) (2017)), in many biofilms, extracellular DNA (eDNA) functions as a structural scaffolding within the EPS, and can help facilitate bacterial adhesion, aggregation, and horizontal gene transfer. Initially, it was assumed that the DNA found within biofilms was merely a remnant of lysed cells, and the first study that showed that eDNA can be a vital, contributing component of bacterial biofilms was done by Whitchurch et al. in 2002 (Whitchurch C. B., Tolker-Nielsen T., Ragas P. C., Mattick J. S. Extracellular DNA required for bacterial biofilm formation. Science. 2002; 295:1487. doi:10.1126/science.295.5559. 1487.). The authors showed that exogenously added deoxyribonuclease (DNase I) was able to inhibit the formation of P. aeruginosa biofilms in vitro without significantly affecting bacterial viability. Additionally, they found that treating established P. aeruginosa biofilms up to 60 h with DNase I led to dispersal. This finding triggered a wave of research into targeting eDNA with various DNases as a means to eradicate biofilm infections. Table 1 summarizes many of the DNases that have been shown to have biofilm-disrupting activity to date.

TABLE 1
DNases that disperse Biofilms

| Enzyme | Summary |
|---|---|
| DNase I | A pancreatic DNase that has been shown to deconstruct the |
| | established biofilms of a wide range of microbes, including P. |
| | aeruginosa, Vibrio cholerae, E. coli, S. pyogenes, Klebsiella |
| | pneumoniae, Acinetobacter baumannii, Aggregatibacter |
| | actinomycetemcomitans, Shewanella oneidensis, S. |
| | heamolyticus, Bordetella pertussis, Bordetella bronchiseptica, |
| | Campylobacter jejuni, H. influenza, B. bacteriovorus, S. |
| | aureus, Enterococcus faecalis, Listeria monocytogenes, |
| | Candida albicans, and Aspergillus fumigatus. |
| DNase | A human DNase found in keratinocytes that has been shown |
| 1L2 | to degrade the established biofilms of P. aeruginosa and |
| | S. aureus. |
| Dornase | A highly purified form of recombinant human DNase I |
| alpha | (rhDNase I) that has been shown to be effective against the |
| | established biofilms of S. aureus and Streptococcus |
| | pneumoniae. |
| λ | A viral DNase that disrupts established V. cholerae biofilms |
| Exo- | |
| nuclease | |
| NucB | A bacterial DNase produced by the marine bacterium, Bacillus |
| | licheniformis, which has been show able to degrade the |
| | established biofilms of multiple bacterial species, including |
| | B. licheniformis, S. aureus, S. epidermidis, Staphylococcus |
| | salivarius, Staphylococcus constellatus, S. Staphylococcus |
| | lugdunesis, Staphylococcu sanginosus, E. coli, Streptococcus |
| | intermedius, Micrococcus luteus, and Bacillus subtilis. |
| Strepto- | A streptococcal DNase that disrupts the established biofilms |
| dornase | of P. aeruginosa. |

As stated in Kuehnast, et al. (T. Kuehnast, F. Cakar, T. Weinhaupl, A. Pilz, S. Selak, M. A. Schmidt, C. Ruter, S. Schild, Comparative analyses of biofilm formation among different Cutibacterium acnes isolates, Int J Med Microbiol 308(8) (2018) 1027-1035), it is becoming increasingly evident that biofilm formation is an important feature for P. acnes pathogenesis of skin diseases and implant-associated infections. P. acnes isolates are characterized by a high genetic heterogeneity, which allows the classification into different phylotypes and sub-types. Kuehnast et al. provided a first comparative analysis of in vitro biofilm formation capacities using a comprehensive collection of P. acnes isolates comprising representatives categorized by phylotypes (IA1, IA2, IB, IC, II and III), IA1 SLST sub-types and anatomical isolation site (skin and implant). In the microtiter plate assay, which employed more stringent washing steps, skin- and implant-derived IA1 isolates showed 2-8-fold higher biofilm formation capacity compared to other phylotypes. In particular, SLST sub-types A1 and A2 exhibit high biofilm formation capacity, which is an interesting finding considering that these sub-types were shown to have a stronger association with mild to severe acne. Microscopic analyses of the biofilm morphologies allowed visualization and evaluation of the three-dimensional biofilm structures. This resulted in a more refined assessment of biofilm formation by diverse P. acnes isolates, with well-structured mature biofilms formed by phylotypes IA1, IB, and IC. Concordantly, these isolates also showed the highest attachment rates to abiotic surfaces. In general, no consistent differences in biofilm formation between skin- and implant-derived isolates of the same phylotype could be observed. A notable exception is the IA1 phylotype, with slightly higher biofilm values of implant-derived isolates compared to skin-derived isolates in both assays. Proteinase K- and DNase I-sensitivity assays revealed that both, eDNA and proteins, are important for initial attachment to abiotic surfaces and that proteins are important structural components of the mature biofilms formed by all phylotypes. In contrast, a phylotype-dependent difference in DNase I-sensitivity of mature P. acnes biofilms could be observed. Taken together, the results indicated that biofilm formation by P. acnes is primarily dictated by the phylotype and to a much lower extent by the anatomical site of isolation.

The impact of DNase I- and proteinase K-treatment was also assessed by Kuehnast et al. in the microtiter plate biofilm assays. Using this high-throughput assay, they were able to test effects of several different enzyme concentrations. However, the assay was limited to IA1 isolates, as only these showed decent biofilm formation in microtiter plates. In contrast to the flow-cell based assay, IA1 biofilms in the microtiter plates were susceptible to both, DNase I- and proteinase K-treatment. In comparison to the mock-treated control significant reductions in the biofilm amount were observed with proteinase K concentrations down to 1.9 μg/ml and DNase I concentrations down to 1.9 ng/ml. Kuehnast et al. therefore, speculated that DNase I-treatment in combination with the more intense washing steps during the microliter plate assay had a stronger negative effect on the adhesive properties of the biofilm, compared to the same treatment performed under the assay conditions inside the microscopy chamber.

### SUMMARY OF THE INVENTION

The present inventor has discovered that if Propionibacterium granulosum is present, the ability of P. acnes to form biofilms is negatively affected. The present inventor has further been able to determine that P. granulosum secretes a protein that disrupts P. acnes biofilms. The P. granulosum protein has been isolated and identified to have DNase activity.

Accordingly, one aspect of the present invention provides for an isolated protein having an amino acid sequence according to SEQ ID NO:2, and functional variants thereof having an amino acid sequence identity of at least 50% to SEQ ID NO: 2 and having at least 80% of the DNase activity of the protein according to SEQ ID NO: 2 in a quantitative assay of deoxyribonuclease activity at pH 7 and 32° C., for use in medicine.

The protein may furthermore be for use in a method for treatment and/or prevention of a disease caused or complicated by infections of one or more biofilm-forming bacteria and/or fungi.

The protein may be for use according to the above, wherein said disease is caused or complicated by infections of Propionibacterium acnes, P. aeruginosa, Vibrio cholerae, E. coli, S. pyogenes, Klebsiella pneumoniae, Acinetobacter baumannii, Aggregatibacter actinomycetemcomitans, Shewanella oneidensis, S. heamolyticus, Bordetella pertussis, Bordetella bronchiseptica, Campylobacter jejuni, H. influenza, B. bacteriovorus, S. aureus, Enterococcus faecalis, Listeria monocytogenes, Candida albicans, Aspergillus fumigatus. Streptococcus pneumonia, B. licheniformis, S. epidermidis, Staphylococcus salivarius, Staphylococcus constellatus, Staphylococcus lugdunesis, Staphylococcus anginosus, E. coli, Streptococcus intermedius, Micrococcus luteus, and Bacillus subtilis.

The protein may be for use according to the above, wherein the disease is a disease of the skin.

The protein may be for use according the above, wherein the disease of the skin is selected from the group consisting of acne vulgaris, candidiasis, bullous impetigo, rosacea and pemphigus foliaceus.

The protein may be for use according to the above, wherein said protein is for use in a method for promoting healing of wounds.

The protein may be for use according to the above, wherein the wounds are selected from diabetic foot ulcers, pressure ulcers, vascular ulcers, ischemic wounds, burn wounds, and surgical wounds.

Furthermore, the present disclosure provides for a pharmaceutical composition comprising the protein according to the above and optionally pharmaceutically acceptable excipients.

The pharmaceutical composition according to the above may further comprise a lipid carrier system and/or an aqueous pH buffer.

According to one embodiment of the pharmaceutical composition according the above, the lipid carrier system comprises lipids in a solid form or in a crystalline form.

Also provided herein is a method for the treatment and/or prevention of a disease caused or complicated by infections of one or more biofilm-forming bacteria and/or fungi, comprising administering a protein or pharmaceutical composition according to the above to a subject affected by said infection. The protein or pharmaceutical composition is preferably administered to the site of the biofilm-forming bacteria and/or fungi in an amount effective to reduce the biofilm.

Said disease may be caused or complicated by infections of Propionibacterium acnes, P. aeruginosa, Vibrio cholerae, E. coli, S. pyogenes, Klebsiella pneumoniae, Acinetobacter baumannii, Aggregatibacter actinomycetemcomitans, Shewanella oneidensis, S. heamolyticus, Bordetella pertussis, Bordetella bronchiseptica, Campylobacter jejuni, H. influenza, B. bacteriovorus, S. aureus, Enterococcus faecalis, Listeria monocytogenes, Candida albicans, Aspergillus fumigatus. Streptococcus pneumonia, B. licheniformis, S. epidermidis, Staphylococcus salivarius, Staphylococcus constellatus, Staphylococcus lugdunesis, Staphylococcus anginosus, E. coli, Streptococcus intermedius, Micrococcus luteus, and Bacillus subtilis.

According to one embodiment of said method, the disease is a disease of the skin.

According to one embodiment of said method, the disease of the skin is selected from the group consisting of acne vulgaris, candidiasis, bullous impetigo, rosacea and pemphigus foliaceus.

According to one embodiment of said method, the method is for promoting healing of wounds. According to a further embodiment, the wounds are selected from diabetic foot ulcers, pressure ulcers, vascular ulcers, ischemic wounds, burn wounds, and surgical wounds.

The protein according to the above may be the P. granulosum DNase PG_1116 having the sequence SEQ ID NO: 2, the homologous DNase from the P. granulosum DSM20700 strain (GenBank accession no. WP_021104654, or the homologous DNase from the P. granulosum TM11 strain (GenBank accession no. ERF66724).

### BRIEF DESCRIPTION OF FIGURES

FIG. 1A. PG_1116 DNase activity on plasmid DNA.

P. acnes genomic DNA was treated for 22 h at 37° C. with (1) PBS, (2) PG_1116 purified protein, or (3) PG_1116 purified protein heat-inactivated for 10 min at 95° C. and run on a 1% agarose el with a (M) molecular weight marker.

FIG. 1B. PG_1116 DNase activity on plasmid DNA.

Plasmid DNA was treated for 5 min at 37° C. with different concentrations of (conc in mg/mL) of DNaseI (D), PG-1116 purified protein (P), or PG-1116 purified protein heat-inactivated for 10 min at 95° C. (PI) in the presence or absence of EDTA. Samples were run on a 1% agarose el with a (M) molecular weight marker.

FIG. 2. Cell-free P. granulosum has DNase activity.

I. The 50 kDa fraction of P. granulosum conditioned cell-free medium has DNase activity.

II. This DNase activity is not further enhanced by MG²⁺.

III. EDTA inhibits the DNase activity

FIG. 3. DNase activity assay

A) Enzyme kinetics graph of DNase I. B) Enzyme kinetics graph of PG_1116. C) Enzyme kinetics graph of NucB. D) Re-plotted graph of enzymatic activity at 25° C., for NucB, from 0-8 minutes. E) Re-plotted graph of enzymatic activity at 25° C., for PG_1116, from 0-8 minutes.

### SEQUENCE LISTING

The following sequences are included in the sequence listing

SEQ ID NO: 1: DNA sequence encoding the isolated protein according to SEQ ID NO: 2.

SEQ ID NO: 2: Isolated protein derived from Propionibacterium granulosum, with DNase activity. This protein is also termed “PG_1116”.

### DETAILED DESCRIPTION OF THE INVENTION

Proteins having DNase activity according to the present invention can be isolated from bacteria of the species Propionibacterium granulosum and/or produced by recombinant DNA techniques well known in the art. The term “isolated” as used herein reflects that the protein is isolated from its natural environment.

The present invention relates to an isolated protein having the amino acid sequence according to SEQ ID NO: 2 and functional variants of this protein that have retained or essentially the same DNase activity as the protein of SEQ ID NO: 2, i.e. the capability to degrade deoxyribonucleic acid (DNA). A functional variant is a protein wherein at one or more positions there have been amino acid insertions, deletions, or substitutions, either conservative or non-conservative, provided that such changes result in a protein whose function as relates to DNase activity is significantly retained. “Significantly” in this context means that the functional variant has at least 80%, such as 85%, 90%, 95%, 100% or more of the DNase activity of the protein according to SEQ ID NO: 2 in a quantitative assay of deoxyribonuclease I (EC 3.1.21.1) activity. The functional variants may be assessed for retained DNase activity e.g. at pH 7 and 32° C. or at pH 6 and 25° C. Such quantitative assays are known in the art and also described in the experimental section below. A functional variant preferably has an amino acid sequence identity of at least 50%, such as 60%, 65%, 70%, 75%, 80%, 85%, 90%, 95%, 96%, 97%, 98%, 99% or 100% with SEQ ID NO: 2.

Accordingly, a functional variant of an isolated protein with an amino acid sequence according to SEQ ID NO: 2 retains its DNase activity, and the ability to disrupt biofilms

By “conservative substitutions” is intended substitutions within the groups Gly, Ala; Val, Ile, Leu; Asp, Glu; Asn, Gln; Ser, Thr; Lys, Arg; and Phe, Tyr.

Such variants may be made using the methods of protein engineering and site-directed mutagenesis which are well known in the art.

When used in medicine, the DNase of the present invention may be administered in the form of a conventional pharmaceutical composition.

The pharmaceutical composition can be in the form of an aqueous solution. An aqueous solution refers to a solution having physiologically or pharmaceutically acceptable properties regarding pH, ionic strength, isotonicity etc. As examples can be mentioned isotonic solutions of water and other biocompatible solvents, aqueous solutions, such as saline and glucose solutions, and hydrogel-forming materials. The aqueous solution can be buffered, such as phosphate-buffered saline, PBS.

The pharmaceutical composition can in addition comprise pharmaceutical acceptable excipients, such as a preservative to prevent microbial growth in the composition, antioxidants, isotonicity agents, colouring agents and the like. In aqueous suspensions the compositions can be combined with suspending and stabilising agents. The pharmaceutical composition may further comprise an additional pharmaceutically active compound, such as an antibiotic.

The colloidal nature of the composition makes it possible to prepare the composition aseptically by using a final sterile filtration step.

In order to form a gel the protein can be preferably formulated with a hydrogel-forming material. Examples of hydrogel-forming materials are synthetic polymers, such as polyvinylalcohol, polyvinylpyrolidone, polyacrylic acid, polyethylene glycol, poloxamer block copolymers and the like; semi-synthetic polymers, such as cellulose ethers, including carboxymethylcellulose, hydroxyethylcellulose, hydroxy-propylcellulose, methylcellulose, methylhydroxypropylcelltalose and ethylhydroxy-ethylcellulose, and the like; natural gums, such as acacia, carragenan, chitosan, pectin, starch, xanthan gum and the like.

It is advantageous to use a hydrogel which is muco-adhesive. In that respect it is particularly useful to use hyaluronic acid and derivatives thereof, cross-linked polyacrylic acids of the carbomer and polycarbophil types, polymers that readily form gels, which are known to adhere strongly to mucous membranes.

It is also advantageous to use block copolymers of the poloxamer type, i. e. polymers consisting of polyethylene glycol and polypropylene glycol blocks. Certain poloxamers dispersed in water are thermoreversible: at room temperature they are low viscous but exhibit a marked viscosity increase at elevated temperatures, resulting in a gel formation at body temperature. Thereby the contact time of a pharmaceutical formulation administered to the relatively warm skin may be prolonged and thus the efficacy of the incorporated DNase may be improved.

The pharmaceutical composition of the invention can be formulated for topical or enteral, that is oral, buccal, sublingual, mucosal, nasal, bronchial, rectal, and vaginal administration.

In one preferred embodiment of the present invention, the route of administration may be topical.

Non-limiting examples of pharmaceutical compositions for topical administration are solutions, sprays, suspensions, emulsions, gels, and membranes. If desired, a bandage or a band aid or plaster can be used, to which the pharmaceutical composition has been added. Tablets, capsules, solutions or suspensions can be used for enteral administration.

Depending on the mode of administration, the pharmaceutical composition will according to one embodiment of the present invention include 0.05% to 99% weight (percent by weight), according to an alternative embodiment from 0.10 to 50% weight, of the protein of the present invention, all percentages by weight being based on total composition.

A therapeutically effective amount for the practice of the present invention may be determined, by the use of known criteria including the age, weight and response of the individual patient, and interpreted within the context of the disease which is being treated or which is being prevented, by one of ordinary skills in the art.

The proteins for use according to the invention can be produced by recombinant DNA technology.

Techniques for construction of plasmids, vectors and expression systems and transfection of cells are well-known in the art, and the skilled artisan will be familiar with the standard resource materials that describe specific conditions and procedures.

Construction of the plasmids, vectors and expression system of the invention employs standard ligation and restriction techniques that are well-known in the art (see generally, e.g., Ausubel, et al, Current Protocols in Molecular Biology, Wiley Interscience, 1989; Sambrook and Russell, Molecular Cloning, A Laboratory Manual 3rd ed. 2001). Isolated plasmids, DNA sequences, or synthesized oligonucleotides are cleaved, tailored, and relegated in the form desired. Sequences of DNA constructs can be confirmed using, e.g., standard methods for DNA sequence analysis (see, e.g., Sanger et al. (1977) Proc. Natl. Acad. Sci., 74, 5463-5467).

Yet another convenient method for isolating specific nucleic acid molecules is by the polymerase chain reaction (PCR) (Mullis et al. Methods Enzymol 155:335-350, 1987) or reverse transcription PCR (RT-PCR). Specific nucleic acid sequences can be isolated from RNA by RT-PCR. RNA is isolated from, for example, cells, tissues, or whole organisms by techniques known to one skilled in the art. Complementary DNA (cDNA) is then generated using poly-dT or random hexamer primers, deoxynucleotides, and a suitable reverse transcriptase enzyme. The desired polynucleotide can then be amplified from the generated cDNA by PCR. Alternatively, the polynucleotide of interest can be directly amplified from an appropriate cDNA library. Primers that hybridize with both the 5′ and 3′ ends of the polynucleotide sequence of interest are synthesized and used for the PCR. The primers may also contain specific restriction enzyme sites at the 5′ end for easy digestion and ligation of amplified sequence into a similarly restriction digested plasmid vector.

As will be evident from the examples below, the inventor has shown that the P. granulosum DNase PG_1116, which is a protein having an amino acid sequence according to SEQ ID NO:2, is significantly more effective in degrading a biofilm produced by P. acnes at a pH of 7, than NucB. The pH on the surface of normal skin is in the range of 4-5.5. However, skin affected by acne normally has a higher pH than unaffected skin, with a mean value for acne patients of pH 6.4, but for some patients reaching pH levels of 10 or higher (Prakash, C. et al 2017 Skin Surface pH in Acne Vulgaris: Insights from an Observational Study and Review of the Literature. J Clin Aesthet Dermatol. 10: 33-39). Consequently, PG_1116 is more efficient than other enzymes used for the same purpose, such as NucB, upon treatment of skin affected by acne to degrade the biofilm produced by P. acnes.

Therefore, the present disclosure provides for an isolated protein having an amino acid sequence according to SEQ ID NO:2, and functional variants thereof having retained DNase activity.

The isolated protein according the above may be for use in medicine. The protein may furthermore be for use in treatment and/or prevention of a disease caused or complicated by infections of one or more biofilm-forming bacteria and/or fungi.

Said disease may be caused or complicated by infections of Propionibacterium acnes, P. aeruginosa, Vibrio cholerae, E. coli, S. pyogenes, Klebsiella pneumoniae, Acinetobacter baumannii, Aggregatibacter actinomycetemcomitans, Shewanella oneidensis, S. heamolyticus, Bordetella pertussis, Bordetella bronchiseptica, Campylobacter jejuni, H. influenza, B. bacteriovorus, S. aureus, Enterococcus faecalis, Listeria monocytogenes, Candida albicans, Aspergillus fumigatus. Streptococcus pneumonia, B. licheniformis, S. epidermidis, Staphylococcus salivarius, Staphylococcus constellatus, Staphylococcus lugdunesis, Staphylococcus anginosus, E. coli, Streptococcus intermedius, Micrococcus luteus, and Bacillus subtilis. These are all biofilm-forming bacteria or fungi.

The protein may be for use according to the above, wherein the disease is a disease of the skin. Said disease of the skin may be selected from the group consisting of acne vulgaris, candidiasis, bullous impetigo, rosacea and pemphigus foliaceus.

Preferably, the protein may be for use in the treatment and/or prevention of acne vulgaris. Furthermore, the protein may be for use in degradation of a biofilm formed by Propionibacterium Acnes (P. Acnes). The protein may further be for use in degradation of a biofilm formed by P. Acnes of the subtype I1A.

It has been shown that biofilms formed on implants such as pacemaker devices, causes biofilm-associated infections. These infections may be difficult to combat, and may lead to surgical wounds not healing properly. In general biofilms are difficult to degrade by the immune system and may thus cause any wound to not heal properly. Okuda et al. (K. I. Okuda, R. Nagahori, S. Yamada, S. Sugimoto, C. Sato, M. Sato, T. Iwase, K Hashimoto, Y. Mizunoe, The Composition and Structure of Biofilms Developed by Propionibacterium acnes Isolated from Cardiac Pacemaker Devices, Front Microbiol 9 (2018) 182) investigated the efficacy of enzymes targeting P. acnes biofilm matrix constituents against biofilm formation by the five isolates. They used DNase I, proteinase K, and dispersin B, which digest DNA, protein, and poly-N-acetyl glucosamine (poly-GlcNAc), respectively, and showed that DNase I significantly inhibited biofilm formation for strains isolated from cardiac pacemaker devices. Therefore, the protein according to the present invention may be for use according to the above, wherein said protein is for use in promoting healing of wounds. Said wounds may be selected from diabetic foot ulcers, pressure ulcers, vascular ulcers, ischemic wounds, burn wounds, and surgical wounds.

Furthermore, the present disclosure provides for a pharmaceutical composition comprising the protein according to the above and optionally pharmaceutically acceptable excipients.

The pharmaceutical composition according to the above may further comprise a lipid carrier system and/or an aqueous pH buffer.

According to one embodiment of the pharmaceutical composition according the above, the lipid carrier system comprises lipids in a solid form or in a crystalline form.

As stated above, the composition above may comprise a pH buffer, preferably a water-based pH buffer. As indicated above, skin affected by acne has a higher pH than unaffected skin. By comprising a pH buffer in the composition, the pH on skin affected by acne may be buffered to a pH that will be disadvantageous for the P. acnes and thus further ameliorate the result of treatment of acne with such a composition. However, care must be taken so that the pH is not lowered to a pH wherein the protein of the present invention becomes less efficient, as is apparent from the experimental section below.

The protein may thus degrade the biofilm, whereas the pH buffer may buffer the pH to a level that may help in healing out the bacterial infection of P. acnes that causes the acne. Thus, the composition according to the present disclosure may have two mechanisms of action. The primary mechanism is efficiently degrading the biofilm, at the higher pH that is normally consistent with skin affected by acne, thereby allowing for the composition to penetrate the comedones associated with acne. The secondary mechanism is buffering the pH, thereby making the growing conditions for P. acnes less optimal. Accordingly, the composition according to the above may be provided for use in medicine. The composition according to the above may be for use in treatment and/or prevention of a disease caused or complicated by infections of one or more biofilm-forming bacteria and/or fungi. The composition according to the above many be for use wherein said disease is caused or complicated by infections of Propionibacterium acnes, P. aeruginosa, Vibrio cholerae, E. coli, S. pyogenes, Klebsiella pneumoniae, Acinetobacter baumannii, Aggregatibacter actinomycetemcomitans, Shewanella oneidensis, S. heamolyticus, Bordetella pertussis, Bordetella bronchiseptica, Campylobacter jejuni, H. influenza, B. bacteriovorus, S. aureus, Enterococcus faecalis, Listeria monocytogenes, Candida albicans, Aspergillus fumigatus. Streptococcus pneumonia, B. licheniformis, S. epidermidis, Staphylococcus salivarius, Staphylococcus constellatus, Staphylococcus lugdunesis, Staphylococcus anginosus, E. coli, Streptococcus intermedius, Micrococcus luteus, and Bacillus subtilis.

The composition according to the above may be intended for use in degradation of a biofilm formed by Propionibacterium Acnes (P. Acnes). The composition according to the above may further be intended for use in degradation of a biofilm formed by P. Acnes of the subtype I1A.

The composition according to the above may be for use wherein the disease is a skin disease. The disease of the skin may be selected from the group consisting of acne vulgaris, candidiasis, bullous impetigo, rosacea and pemphigus foliaceus

The composition according to the above may further be for use in promoting healing of wounds. The wound can be selected from diabetic foot ulcers, pressure ulcers, vascular ulcers, ischemic wounds, burn wounds, surgical wounds.

The lipid carrier system may comprise lipids in a solid form or in a crystalline form. Preferably the lipids are in crystalline form. The lipids in crystalline form may for instance be monoglycerides. In general, it has previously been noted that enzymatic activity is at least partly inhibited by presence of lipids. Many of the enzymes according to the prior art are sensitive to both pH and presence of lipids, as the enzymes are inactivated. This is problematic also as sebum will be present on the skin of a patient with acne skin. However, by using the protein of the present invention this problem is overcome. Said protein is not inactivated by the presence of lipids, and can thus be active with the lipids in the formulation, and on the skin even when sebum is present.

### Amino Acid Sequence Identity

The percent identity between two amino acid sequences is determined as follows. First, an amino acid sequence is compared to, for example, SEQ ID NO:2 using the BLAST 2 Sequences (B12seq) program from the stand-alone version of BLASTZ containing BLASTN version 2.0.14 and BLASTP version 2.0.14. This stand-alone version of BLASTZ can be obtained from the U.S. government's National Center for Biotechnology Information web site at ncbi.nlm.nih.gov. Instructions explaining how to use the B12seq program can be found in the readme file accompanying BLASTZ. B12seq performs a comparison between two amino acid sequences using the BLASTP algorithm. To compare two amino acid sequences, the options of B12seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\B12seq -i c:\seq1.txt -j c:\seq2.txt -p blastp -o c:\output.txt. If the two compared sequences share homology, then the designated output file will present those regions of homology as aligned sequences. If the two compared sequences do not share homology, then the designated output file will not present aligned sequences. Once aligned, the number of matches is determined by counting the number of positions where an identical nucleotide or amino acid residue is presented in both sequences.

The percent identity is determined by dividing the number of matches by the length of the sequence set forth in an identified sequence followed by multiplying the resulting value by 100. For example, if a sequence is compared to the sequence set forth in SEQ ID NO:A (the length of the sequence set forth in SEQ ID NO:A being 10) and the number of matches is 9, then the sequence has a percent identity of 90% (i.e., 9/10*100=90) to the sequence set forth in SEQ ID NO:A.

### Examples

### Strains Used and Culture Conditions

Propionibacterium granulosum DSM 20700 was obtained from the German collection of microorganisms. The strain was grown anaerobically at 37° C. either on solid medium on Blood-Agar Petri dishes or in liquid medium in BHI supplemented with 2 g/L glucose. Planktonic cultures were grown with shaking (200 rpm) whereas biofilms were grown in static flasks with medium change every other day.

P. granulosum Genome Sequencing

P. granulosum genomic DNA was isolated from a liquid culture using GenElute™ Bacterial Genomic DNA Kit (Sigma-Aldrich Chemie GmbH, Steinheim, Germany). Sequencing of P. granulosum was performed on Illumina HiSeq 2000 as paired-end 2×100 bp providing an approximate 100× overall raw base pair coverage. The raw sequencing data was quality controlled using FastQC version 0.10.1. (http://www.bioinformatics.bbsrc.ac.uk/projects/fastqc). SOAPDenovo version 1.05 (Li et al. 2010. De novo assembly of human genomes with massively parallel short read sequencing.” Genome Res 20(2): 265-272), was used to perform de novo assembly using the raw reads. Standard parameters for paired-end reads were used. The K-mer setting generating scaffold sequences with the largest N50 was 77. For gap closure, K-mer error correction was performed on the raw reads using Quake version 0.3.4 (Kelley et al. 2010. Quake: quality-aware detection and correction of sequencing errors.” Genome Biol 11(11): R116). The total genome size was 2,488,918 bp, the longest sequence was 702,365 bp and the N50 359,503 bp. Before annotation the raw contig sequences were trimmed (≥300 bp) and reverse sorted according to sequence length. The gene annotation was performed using the CloVR pipeline version 1.0-RC4 (Angiuoli et al. 2011. CloVR: A virtual machine for automated and portable sequence analysis from the desktop using cloud computing. Bmc Bioinformatics 12). Specifically, rRNA annotation was performed using RNAmmer (Lagesen et al. 2007. RNAmmer: consistent and rapid annotation of ribosomal RNA genes. Nucleic Acids Research 35(9): 3100-3108). tRNA annotation was performed using tRNAscan-SE (Lowe et al. 1997. tRNAscan-SE: a program for improved detection of transfer RNA genes in genomic sequence. Nucleic Acids Res 25(5): 955-964) and prediction of protein coding regions (CDS) was performed using Glimmer (Delcher et al. 2007. Identifying bacterial genes and endosymbiont DNA with Glimmer. Bioinformatics 23(6): 673-679). Functional annotation of CDS was performed using the IGS annotation engine (http://ae.igs.umaryland.edu/cgi/ae_pipeline_outline.cgi). PG_1116 is a predicted protein of 939 amino-acids (SEQ ID NO:2) with a molecular mass of 96 kDa, it was attributed by BLAST homology to COG2347 containing predicted extracellular nuclease. PG_1116 contains a DNaseI domain comprising putative catalytic, active, DNA binding, phosphate binding and Mg binding sites at its C-terminus and a Lamin-tail domain at its N-terminus. A TAT peptide for protein secretion via the Sec-pathway was also identified at its far N-terminus end. Two further domains not yet well defined are also present: YhcR-OBF domain corresponding to a subfamily of OB-fold domains that could be important for recognition of specific patterns and a non-specific fungal domain of unknown function.

Further BLAST analysis revealed that PG_1116 sequence had 97% identity with the corresponding published genome sequence of P. granulosum DSM20700 and 94% with P. granulosum TM11. The protein is also conserved in other Propionibacteria species as P. avidum (61% identity on 87% cover, the fungal domain is missing). Interestingly the protein was absent of all the P. acnes annotations present in the NCBI database, the maximal cover was 35% and always included only one of the domains described. This particular domain arrangement might therefore be important for a potential anti-P. acnes biofilm activity of the protein.

### PG_1116 Overexpression and Purification

The PG_1116 gene was amplified by PCR and cloned in pET-ZZ1a using restriction sites corresponding NcoI and HindIII. PG_1116 was overexpressed and purified using NiNTA and Q-sepharose (TEV-cleaved) columns and eluted in buffer 50 mM NaP 8.0, 500 mM NaCl, 20 mM Imidazole. Aliquots of the protein at a concentration of 0.5 to 1 mg/mL were frozen at −80° C.

### DNase Activity Test

P. acnes DNA (1 μg) or plamid DNA (1.5 μg) was diluted in 20 mM TrisHCl pH 7.4 supplemented with 2 mM CaCl₂ and 2 mM MgCl₂, and others as stated. Incubation with or without 6 μg of the appropriate enzyme: DNaseI, PG_1116 or PG_1116 heat inactivated for 10 min at 95° C. was carried out at 37° C. in a water bath. The reactions were stopped at appropriate time by the addition of 6×DNA Loading Dye buffer (Thermo Scientific™) containing EDTA. Aliquots (5 μL) of the different samples and molecular weight marker (GeneRuler 1 kb DNA ladder Thermo Scientific™) were run on 1% agarose mini-gels, under a constant current of 100 V, for 40 min. DNA was revealed using GelRed™ Nucleic Acid Gel Stain (Biotium) and the Gel Doc™ imager (BioRad).

P. acnes Biofilms Cultures and Disruption Tests

A 48 h planktonic pre-culture of P. acnes IB was diluted 5% v/v in BHI supplemented with 2 g/L glucose and 2 mL was dispended in each well of 24 well plates (Thermo Scientific™ Nunc™ Non-Treated Multidishes 144530). The plates were incubated at 37° C. under static anaerobic conditions. The medium was renewed every 2 days. The effect of different substances was tested on pre-existing 6-days old biofilms by replacing the medium with appropriate dilution of the substance on day 6. Any following incubation was done in semi-aerobic condition.

To test whether the PG_1116 DNase was indeed the effector protein of P. granulosum supernatants able to disrupt P. acnes biofilms, the biofilms were first grown for 6 days and then incubated them with PG_1116 in different conditions. The biofilms were incubated for 1 or 2 hours with PBS, DNaseI, PG_1116, or protein buffer. PG_1116, as well as DNaseI, were capable of disrupting 6-days old biofilms of P. acnes and this activity was also impaired by the addition of EDTA. The biofilm incubated with PG_1116 was disrupted to a higher degree than the biofilm incubated with DNaseI at both timepoints studied, and in particular after 2 h, where the biofilm incubated with PG_1116 was barely detectable.

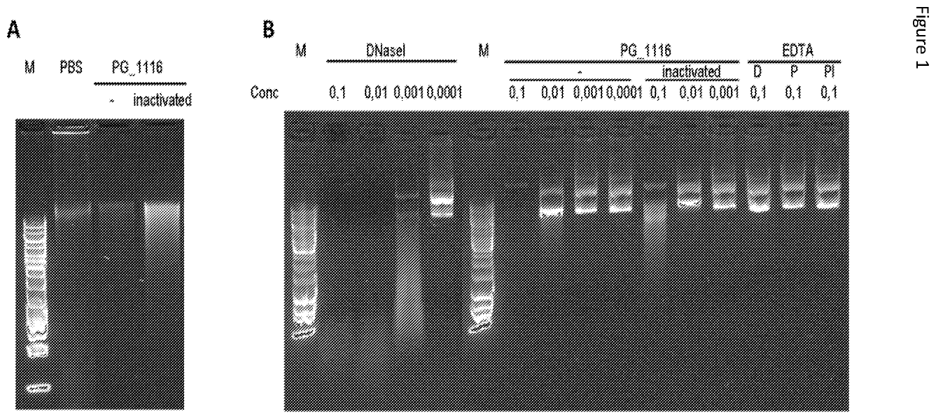

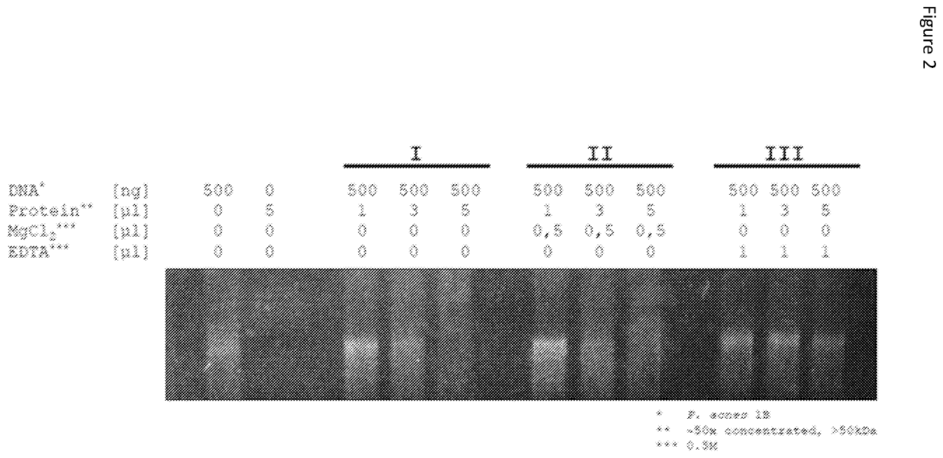

Interestingly the incubation of P. granulosum conditioned culture medium showed DNase like activity and disrupted DNA (FIG. 1B). Likewise co-incubation of P. granulosum conditioned culture medium with P. acnes biofilm resulted in disruption of the biofilm.

PG_1116 Impedes Biofilm Formation from P. acnes

To test whether an exposition to the PG_1116 protein would prevent P. acnes cells to form biofilms, planktonic cultures of P. acnes was grown and exposed to PG_1116 for different times (30 min, 1 h and 2 h), washed and then treated as precultures for biofilm formation. Though all the bacteria were then able to form thin biofilms at the bottom of the flasks, the biofilms formed by the bacteria having been exposed to PG_1116 even for the shortest period of time (30 min) were much weaker than the biofilms formed by bacteria previously exposed to PBS or buffer. Furthermore, if the cells were not washed but just diluted after exposition and before biofilms formation, no biofilm formation could be seen from cells exposed to PG_1116 whereas cells exposed to buffer alone formed next to normal biofilms.

### Comparison of Biofilm-Degrading Activity of PG_1116 and NucB in Different Culture Environments

The cutaneous isolate P. acnes strain KPA171202 was used as a reference strain for all experiments (The complete genome sequence of Propionibacterium acnes, a commensal of human skin. Brüggemann H, Henne A, Hoster F, Liesegang H, Wiezer A, Strittmatter A, Hujer S, Dürre P, Gottschalk G. Science. 2004 Jul. 30; 305(5684):671-3). Bacteria were initially cultured on anaerobic blood agar plates under anaerobic conditions. Plate-grown bacteria were further grown as liquid cultures in brain heart infusion broth (BHI). These pre-cultures were used as inoculum for main cultures grown for either 24 or 48 h anaerobically. Biofilm cultures were grown in T-25 cell culture flasks (Sarstedt, Nümbrecht, Germany) with 10 ml broth and incubated for seven days with medium change every alternate day (Transcriptomic analysis of Propionibacterium acnes biofilms in vitro. Jahns A C, Eilers H, Alexeyev O A. Anaerobe. 2016 December; 42:111-118).

### Comparison of Biofilm-Degrading/Dispersal Activity of NucB and PG_1116 in Culture Medium

After seven days of incubation, PG_1116 and NucB protein with the concentration of 0.1 mg/mL (equal molar ratio) were added and further incubated for 24 h. The effects of PG_1116 and NucB on P. acnes biofilm in culture medium were studied after 24 h. The biofilm incubated with PG_1116 was estimated to be three times smaller as compared with NucB.

Comparison of Biofilm-Degrading/Dispersal Activity of NucB and PG_1116 in Culture Medium Complemented with Artificial Sebum

After seven days of incubation, a 5% sebum emulsion consisting of 150 mg sebum (Pickering Laboratories, Inc., Mountain View, Calif., USA), 10% gum Arabic and 20 mM Tris-HCL was added to the flasks with the intention to mimic hair follicle environment. After addition of sebum, PG_1116 and NucB protein with the concentration of 0.1 mg/mL (equal molar ratio) was added to each flask and incubated for 24 h. After 24 h of incubation, the biofilm degrading/dispersal activity of PG_1116 and NucB in sebum-like environment was compared. The effects of PG_1116 and NucB incubation with P. acnes biofilm in sebum emulsion were studied after 24 h. The biofilm incubated with PG_1116 was estimated to be from three to four times smaller as compared with NucB.

DNase Activity of PG_1116 on P. acnes Biofilm

To study whether PG_1116 DNase activity is related to biofilm degradation, a known enzyme inhibitor (EDTA) can be used. PG_1116 and PG_1116 complemented with EDTA were incubated with P. acnes biofilm in culture medium for 2 h. The biofilm-degrading/dispersal activity of PG_1116 was inhibited when EDTA was added, thus PG_1116 biofilm degrading/dispersal activity is due to DNase activity.

### Analytical Assay for the Determination of DNase Activity

TABLE 2
Chemicals used in the study-

| Name | Manufacturer | Product number |
|---|---|---|
| DNAse 1 recombinant | Roche | 04716728001 |
| Sodium Acetate Trihydrate 500 g | Sigma Aldrich | S8625 |
| Magnesium sulfate heptahydrate 500 g | Sigma Aldrich | M1880 |
| Deoxyribonucleic acid sodium salt from | Sigma Aldrich | D3664 |
| calf thymus | | |
| Deoxyribonuclease 1 from bovine | Sigma Aldrich | D4263 (IVL) |
| pancreas | | |
| HEPES | Sigma Aldrich | |
| Glycerol BioXtra, ≥99% (GC) | Sigma Aldrich | H3375 (25G) |
| Sodium chloride BioXtra | Sigma Aldrich | G6270 (IL) |
| Tris(2-carboxyethyl)phosphine | Sigma Aldrich | S7653 (250G) |
| hydrochloride | | |
| Sodium Acetate Trihydrate 500 g | Sigma Aldrich | |
| Magnesium sulfate heptahydrate 500 g | Sigma Aldrich | |

TABLE 3
Raw materials used for the manufacturing of batch ISM18201(Placebo).

| Compound | Zelmic number | % |
|---|---|---|
| Glycerol monolaurate | CH0729 | 7.0 |
| Glycerol monomyristate | CH0730 | 21.0 |
| Lactic acid | CH0736 | 1.0 |
| NaOH | CH0804 | Used for pH adjustement* |
| Glycerol | CH0852 | 5.0 |
| Water | 20180911 | 66.0 |

*pH adjusted to 5.03

TABLE 4
PG_1116 And NucB Formulations.

| Product name | Concentration | Batch/ID nmbers | Storage buffer |
|---|---|---|---|
| psfPG1116-c001 | 1 mg/ml | psfPG1116-h001 | 20 mM HEPES, |
| | | | 300 mM NaCl, |
| | | | 10% glycerol, |
| | | | 2 mM |
| | | | TCEP, pH 7.5 |
| NucB | 0.2 mg/ml | MBS1153257 | Tris-based buffer, |
| | | | 50% glycerol |

A simple method for measurement of enzymatic activity was set up, based on the procedure developed by Sigma Aldrich. In this assay the DNase catalyses the degradation of DNA according to the reaction below:

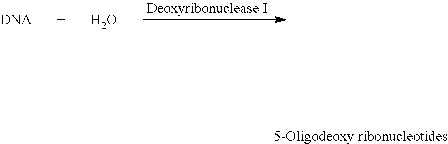

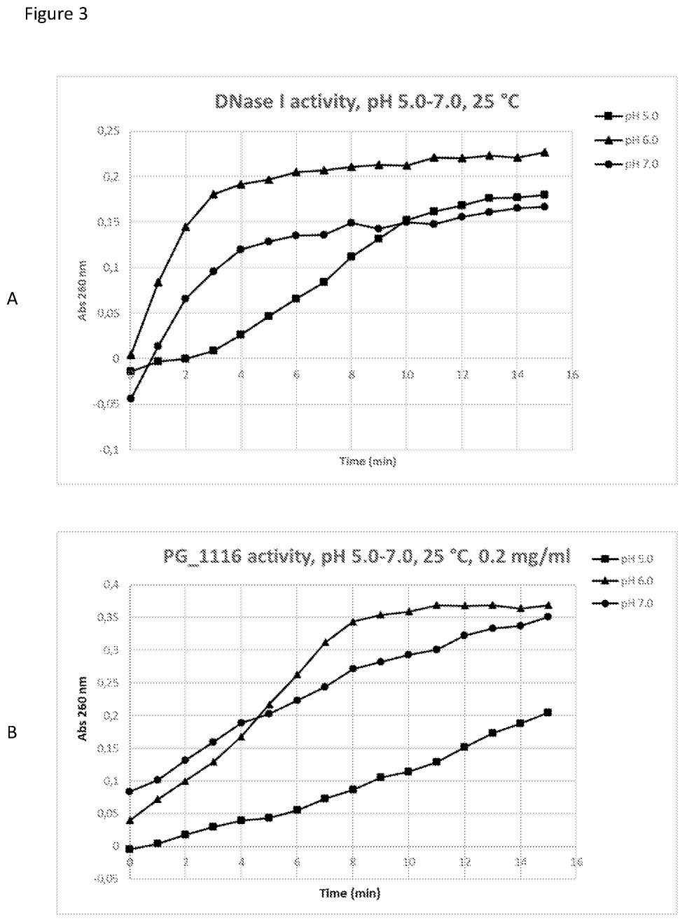

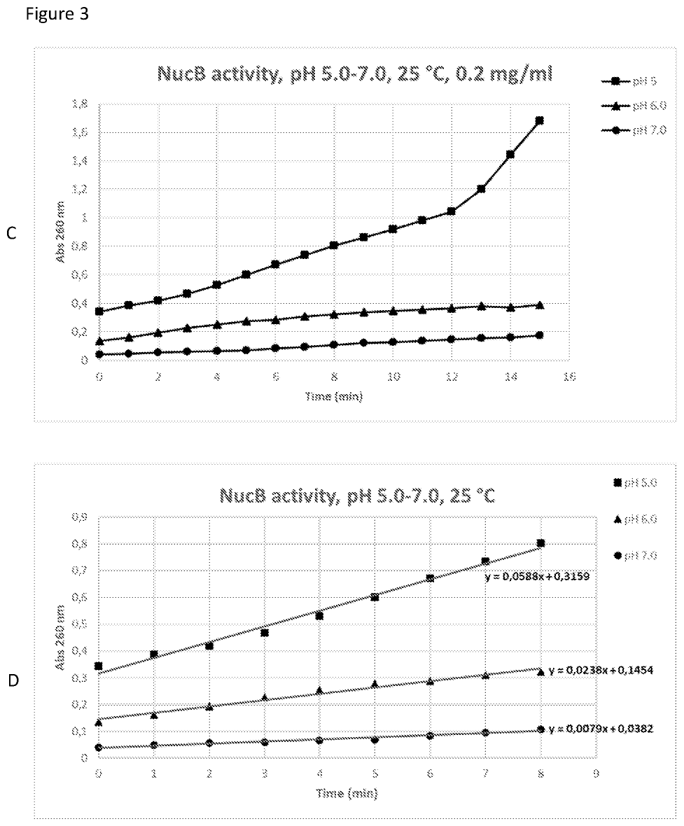

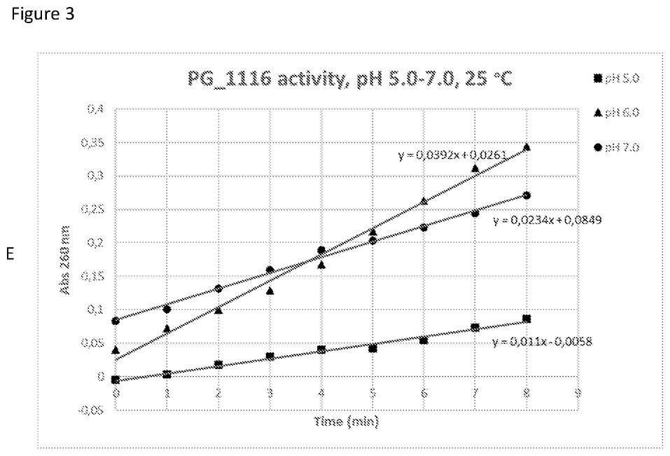

In the first experiment (FIG. 3A) three different incubation buffers were prepared in a falcon tube by varying pH of the acetate buffer (pH 5.0, 6.0 and 7.0) but keeping all other parameters constant. The volume of each added ingredient to the incubation buffer were as follows:
1.25 ml of Sodium Acetate buffer (pH 5.0, 6.0, 7.0) (10%)
0.625 ml MgSO4 (5%)
9.125 ml Purified Water (73%)
1.5 ml DNA solution (added at the end once pH of the buffer was adjusted). DNA solution was reconstituted according to the protocol (to a concentration of 0.33 mg/ml)

DNase from Sigma was reconstituted with 1 ml of 0.85% NaCl solution and further diluted 1:5 with 0.85% NaCl immediately before the use. Blank sample was prepared with each incubation buffer by mixing 100 μl of 0.85% NaCl solution with 500 μl of the incubation buffer (reagent cocktail according to the protocol). The UV spectrophotometer was zeroed with the blank before the actual measurement of the DNase reaction started. For this measurement 100 μl of the DNase was mixed with 500 μl incubation buffer containing DNA (in a quartz cuvette) and the measurements were recorded at every minute during a period of 15 minutes. The experiment was performed at room temperature (˜25° C.). For each buffer (pH 5.0-7.0) the measured absorbance at 260 nm was plotted against the time, see FIG. 3A) for a graph representing enzyme kinetics.

It can be seen from the graph that substrate is consumed after 6 minutes in the buffers with a pH 6 and 7 (flattening curve). The enzymatic reaction is slightly slower in the buffer with a pH 5 by visual assessment of the curve. However, this experiment was performed with the aim of establishing an assay in the lab that can be used for the analysis of PG_1116 and NucB, therefore no further data analysis was performed, it was concluded that the assay was fit for its purpose for further screening of enzyme activities.

### The Effect of pH on Enzymatic Activity in PG_1116 and NucB

In this experiment the activities of PG_1116 and NucB were assessed at different pH's. The experimental work was conducted as described under “Analytical assay for the determination of DNase activity” above. One major difference in this experiment was that due to different concentrations of PG_1116 and NucB the dilutions of the enzyme and preparation of blank samples were prepared as described below:

PG_1116 Blank sample−100 μl of 0.85% NaCl+500 μl Incubation buffer
PG_1116 Sample (1 mg/ml)−diluted 1:5 to 0.2 mg/ml with 0.85% NaCl before mixing with the incubation buffer. Final reaction buffer contains 100 μl of 0.2 mg/ml
PG_1116+500 μl Incubation buffer
NucB Blank Sample (0.2 mg/ml)−100 μl of NucB Storage buffer+500 μl Incubation buffer
NucB sample−100 μl of NucB at 0.2 mg/ml+500 μl Incubation buffer

For the plots of enzymatic activity for both proteins, see FIGS. 3B and 3C.

By visually observing the curves for PG_1116 it can be clearly seen that the optimum activity is achieved at pH 6.0. This curve is also flattening after 8 minutes which shows that the substrate is consumed at this stage. Enzymatic activity was also calculated according to the protocol from Sigma in order to assess activities more accurately. The graphs were re-plotted from 0 to 8 minutes, in order to exclude the part when substrate is consumed for the pH 6.0. FIGS. 3D and 3E show replotted graphs and Table 2 shows calculation of enzyme activity for each enzyme (NucB and PG_1116). The plot of NucB activity is looking slightly different, as no flattening of the curve can be seen. The enzymatic reaction is distinctively slower at higher pH and background absorbance is higher compared to the starting absorbance in PG_1116. There are several different parameters that could affect enzymatic rate and its measurements, however no clear conclusion can be made at this stage with regards to higher absorbance detected at the start. One possible explanation is that due to lower purity profile of NucB (85% pure) there are process related impurities present in the sample that are interfering with measurements at 260 nm. If the process related impurities contain high levels of plasmid DNA fragments, then this could also have an effect on the reaction as the concentration of substrate which is DNA solution is then increased. The starting absorbance at 0 minutes is also different between the different pH's, more obvious in FIG. 3C. The fact that the measurement reading also takes few seconds, it is possible that during this time the reaction has already started and the actual reading at 0 minutes is slightly higher than what the true value is at this time point.

For each replotted curve a linear regression analysis was performed and the slopes (speed/rate of enzymatic reaction) of each regression line were compared. From the data analysis (Table 2) and FIGS. 3D-3E it can be seen that the highest rate for NucB is obtained at pH 5.0, while for the PG_1116 the highest rate is obtained at pH 6.0. At pH 7.0 PG_1116 has a significantly higher activity than NucB. The comparison of activities is also summarized in Table 5 for a better overview of the differences between the different incubation conditions.

TABLE 5
Enzymatic activity in Units/mg

| Enzyme | Temptrature | pH | Units/mg |
|---|---|---|---|
| PG_1116 | 25° C. | 5.0 | 333 |
| | | 6.0 | 1188 |
| | | 7.0 | 709 |
| NucB | 25° C. | 5.0 | 1781 |
| | | 6.0 | 721 |
| | | 7.0 | 239 |

For more details on the calculation of enzyme activity, see an example of calculation below.

Calculation of enzymatic activity, example NucB, 25° C., pH 5.0
The slope is 0.0588 at the pH 5.0. Slope=ΔA260/min
ΔA260 of 0.001/minute/ml=1 unit, according to Sigma's procedure
Units in our sample=0.0588/0.001=58.8 units/ml
Since the concentration of 0.2 mg/ml is diluted 6 times with the reagent cocktail, the final concentration in the reaction buffer is 0.033 mg/ml protein
Activity per mg is equal to 58.8/0.033=1781 Units/mg

### The Effect of Temperature on Enzymatic Activity

In order to investigate the effect of temperature on enzymatic activity a new experiment was designed with the aim of further optimizing the conditions for an optimum activity for both PG_1116 and NucB. In this case a temperature of 32° C. was the most interesting one, as this is the temperature of the surface of the skin. The experimental work was performed as described under “Analytical assay for the determination of DNase activity” above, apart from few minor deviations which are described below:
Only 6 measurements were taken
Cuvette with a final reaction buffer was incubated in a heat cabinet at 32° C. between the measurements, hence it was more practical to reduce the number of measurements

Results from this experiment are presented in Table 6.

TABLE 6
Enzymatic activity in Units/mg

| Enzyme | Temptrature | pH | Units/mg |
|---|---|---|---|
| PG_1116 | 32° C. | 5.0 | 327 |
| | | 6.0 | 503 |
| | | 7.0 | 403 |
| NucB | 32° C. | 5.0 | 2521 |
| | | 6.0 | 867 |
| | | 7.0 | 145 |

The effect of temperature had a positive effect on NucB activity, apart from pH 7.0 which ended up having a slightly lower activity then observed at the pH 7.0, 25° C. The activity at pH 5.0 was significantly higher at the higher temperature, 2521 units/mg at 32° C. compared to 1781 units/mg at the 25° C. The activity of PG_1116 was on the other hand very similar at pH 5.0, 32° C., compared to pH 5.0 at 25° C.; 327 compared to 333 Units/mg. The activity at pH 6.0 and 7.0 was lower at the higher temperature. The aim of this experiment was however to compare activity of NucB and PG_1116 at the skin temperature of 32° C., in a same assay. The most surprising and interesting outcome of this evaluation is the approximately 3 times higher activity observed at the pH 7.0 for PG_1116, compared to NucB. Since the skin affected by acne has a slightly higher pH than a normal skin, this data could be used as a basis for further evaluation of PG_1116 for use in acne treatment.

As the aim of this study was to assess potential of PG_1116 for use in acne treatment, not much emphasis has been put on reproducibility of the method. The two proteins were compared in a same assay on a same day, keeping all other parameters constant, however between the assays performed on a different days the data could vary. One parameter that could have impact on the measurements is the concentration of DNA substrate that can vary between the different vials. However, the effect of this parameter is considered minimal, since the assay is used in routine analysis and each vial is supposed to contain 1 mg of DNA, as labelled on the vial. For the future analysis and a more thorough investigation it is recommended to perform a check of the DNA concentration according to the Sigma protocol and mini qualification of the assay.

### CONCLUSIONS

The most interesting outcome of this evaluation study is the higher activity of PG_1116 at pH 7.0, compared to a commercially available NucB. The activity was approximately 3 times higher at both temperatures, 25 and 32° C. On the other hand, at pH 5.0, 25° C., NucB had 5 times higher activity then PG_1116 and at 32° C., the activity seen in NucB was nearly 8 times higher. However, due to the fact that pH of the skin affected by acne is slightly higher (>6.5) the activity at a pH of 7 is more relevant for the assessment of activity.

1. An isolated protein having an amino acid sequence according to SEQ ID NO:2, and functional variants thereof having an amino acid sequence identity of at least 50% to SEQ ID NO: 2 and having at least 80% of the DNase activity of the protein according to SEQ ID NO: 2 in a quantitative assay of deoxyribonuclease activity at pH 7 and 32° C., for use in medicine.

2. A method for treatment and/or prevention of a disease caused or complicated by infections of one or more biofilm-forming bacteria and/or fungi comprising administering an effective amount of a protein according to claim 1 to a subject in need thereof.

3. The method according to claim 2, wherein said disease is caused or complicated by infections of Propionibacterium acnes, P. aeruginosa, Vibrio cholerae, E. coli, S. pyogenes, Klebsiella pneumoniae, Acinetobacter baumannii, Aggregatibacter actinomycetemcomitans, Shewanella oneidensis, S. heamolyticus, Bordetella pertussis, Bordetella bronchiseptica, Campylobacter jejuni, H. influenza, B. bacteriovorus, S. aureus, Enterococcus faecalis, Listeria monocytogenes, Candida albicans, Aspergillus fumigatus. Streptococcus pneumonia, B. licheniformis, S. epidermidis, Staphylococcus salivarius, Staphylococcus constellatus, Staphylococcus lugdunesis, Staphylococcus anginosus, E. coli, Streptococcus intermedius, Micrococcus luteus, and Bacillus subtilis.

4. The method according to claim 3, wherein the disease is a disease of the skin.

5. The method according to claim 4, wherein the disease of the skin is selected from the group consisting of acne vulgaris, candidiasis, bullous impetigo, rosacea and pemphigus foliaceus.

6. The method according to claim 2, wherein said protein is for use in promoting healing of wounds.

7. The method according to claim 6, wherein the wounds are selected from diabetic foot ulcers, pressure ulcers, vascular ulcers, ischemic wounds, burn wounds, and surgical wounds.

8. A pharmaceutical composition comprising the isolated protein or functional variant thereof according to claim 1 and optionally pharmaceutically acceptable excipients.

9. The pharmaceutical composition according to claim 8, further comprising a lipid carrier system and/or an aqueous pH buffer.

10. The pharmaceutical composition according to claim 9, wherein the lipid carrier system comprises lipids in a solid form or in a crystalline form.
